(19) United States
(12) Patent Application Publication
(10) Pub. No.: US 2008/0020074 A1
(43) Pub. Date: Jan. 24, 2008

(54) Use of a cosmetic compound for promoting skin depigmentation

(75) Inventor: Olivier Courtin, Boulogne Billancourt (FR)

Correspondence Address: ROTHWELL, FIGG, ERNST & MANBECK, P.C., 1425 K STREET, N.W., SUITE 800, WASHINGTON, DC, 20005, US

(73) Assignee: Laboratoires Clarins, Neuilly Sur Seine (FR)

(21) Appl. No.: 11/525,226

(22) Filed: Sep. 22, 2006

(30) Foreign Application Priority Data
Jul. 21, 2006 (FR) 06/06648

(51) Int. Cl. A61K 36/28; A61K 36/73

(52) U.S. Cl. 424/764; 424/765

(57) ABSTRACT

The present invention relates to the use of an extract of Alchemilla vulgaris in a depigmenting cosmetic composition. The present invention also relates to a cosmetic treatment method for depigmenting the skin, by application to the skin of a cosmetic composition containing an extract of Alchemilla vulgaris.

### BACKGROUND OF THE INVENTION

1. Field of the Invention

The present invention relates to the use of a cosmetic composition for promoting skin depigmentation, for example for removing or reducing age spots.

2. Description of the Background Art

Skin pigmentation is related to the presence of specialized cells, melanocytes. The latter represent approximately 5% of the cells of the basal area of the epidermis. They have dendritic extensions which allow them to come into contact with several keratinocytes. Melanocytes have one main function, melanin synthesis or melanogenesis.

Melanogenesis is mainly stimulated by UVA radiation and UVB radiation of the solar spectrum. An increase in the synthesis of melanin pigments, in dendricity and in the number of melanocytes results therefrom. UV radiation which penetrates to the basal area of the epidermis can act directly on the melanocytes or indirectly by stimulating the production of melanogenic agents by keratinocytes (endothelin-1, nitrogen monoxide, etc.).

Melanin is synthesized and stored in specialized vesicles, called melanosomes. Melanosomes are conveyed from the center of the cell to the end of the dendrites, where they may be transferred to the keratinocytes. Melanosome release by melanocytes and phagocytosis by keratinocytes are induced by UV radiation and modulated by numerous factors.

Melanin formation generally takes place continuously and harmoniously in the skin and the hair. However, under certain conditions or under the effect of certain factors, pigmentation disturbances can occur and can cause hyperpigmentation. Several mechanisms may be the cause of this, the main two being increased activity of the melanocytes or melanocyte proliferation. Melanocyte hyperactivity is the cause of ephelides, commonly called freckles, and of chloasma, also call pregnancy mask. Melanocyte proliferation is the cause of senile lentigo, also called age spots. The brown spots appear with age, on the back of the hands, the forearms and the face, generally affecting exposed areas. Histologically, senile lentigo exhibits hyper-pigmentation of the basal area of the epidermis with an increase in the number of melanosomes.

These age-related spots are particularly poorly tolerated and many individuals are tempted to make them disappear. Cosmetology proposes numerous depigmenting cosmetic products which generally act on tyrosinase, an essential enzyme in melanin synthesis. Thus, in patent FR 2 787 710, the applicant had proposed a lightening cosmetic composition in which the active agents inhibit tyrosinase. Certain molecules with depigmenting properties also exist, but the side effects are too toxic for these molecules to be used in cosmetology.

Since depigmenting products are highly prized by, firstly, elderly individuals and by all Asian women for whom whiteness of the skin is essential, the use of a depigmenting composition which is both effective and has no harmful side effects is necessary.

A new receptor involved in melanosome transfer from the melanocyte to the keratinocyte has just been demonstrated; it is the keratinocyte growth factor receptor (hereinafter called KGFR). This receptor, which is activated by keratinocyte growth factor (hereinafter called KGF), is involved in the phagocytosis of melanosomes at the end of the melanocyte dendrites (J Invest Dermatol 125: 1190-1199, 2005).

### SUMMARY OF THE INVENTION

The applicant has demonstrated the inactivation of the KGFR with an extract of Alchemilla vulgaris. Since melanosome transfer from the melanocyte to the keratinocyte by phagocytosis is decreased by the action of Alchemilla vulgaris, the keratinocyte is less rich in melanin, the skin becomes depigmented, the age spots are removed, and the complexion is lightened and more homogeneous. The extract of Alchemilla vulgaris can also be used preventively. This is because it makes it possible to prevent the appearance of pigmentation spots of pathological origin such as chloasma, or natural origin such as ephelides or senile lentigo.

The present invention therefore relates to the use of an extract of Alchemilla vulgaris in a cosmetic or dermatological depigmenting composition.

The present invention also relates to the use of an extract of Alchemilla vulgaris in a cosmetic or dermatological composition, as a depigmenting agent.

### BRIEF DESCRIPTION OF THE INVENTION

The examples refer to the following figures, in which:

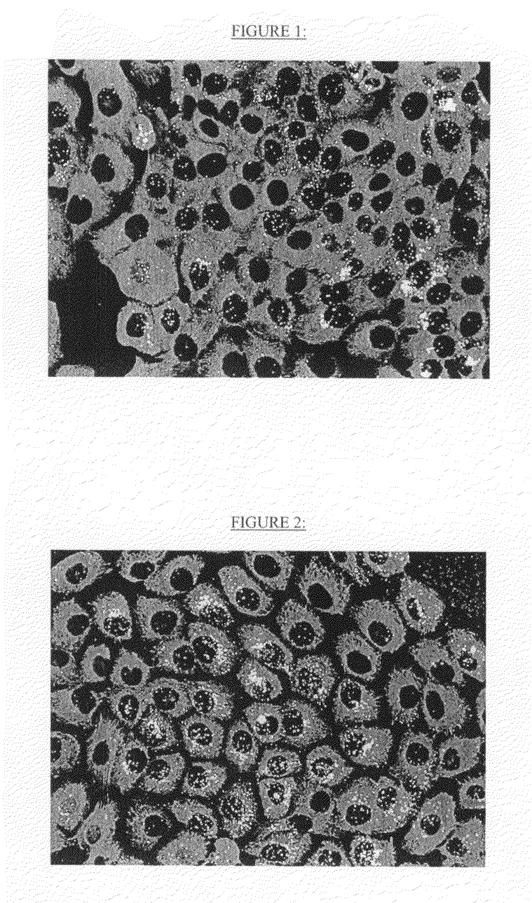

FIG. 1 represents the nontreated control,

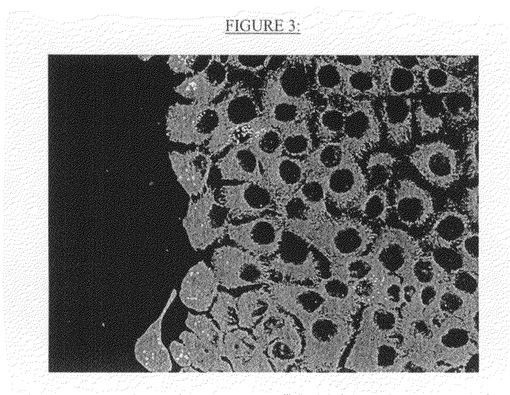

FIG. 2 represents the incorporation of fluorescent beads into the control treated with KGF,

FIG. 3 represents the incorporation of fluorescent beads into the cells treated with KGF and with the extract of Alchemilla vulgaris.

### DETAILED DESCRIPTION OF THE INVENTION

Alchemilla vulgaris is already used in cosmetology as a cleansing agent, but also for its astringent or anti-obesity properties. The use of Alchemilla vulgaris has also been described for repigmenting whitish cicatricial lesions in patent U.S. Pat. No. 4,054,649. Surprisingly, the applicant has demonstrated the fact that certain extracts of Alchemilla vulgaris have the property of promoting skin depigmentation and/or of preventing the appearance of a pigmentation.

Alchemilla vulgaris is a hardy perennial, 20 to 40 cm high, with very light green leaves which are rounded and edged with dentate rounded lobes. Its flowers, which bloom in summer, and which are minute, green and not very bright, are devoid of petals.

Lady's mantle takes its name from the use made of it by alchemists, who collected, as “heavenly water” for preparing the philosopher's stone, the pinkish color at the edge of the leaves and the dewdrops which gather at the center of the latter.

Advantageously, the extract which can be used in the context of the present invention is derived from the whole plant and it is obtained by extraction by means of a solvent such as water, an alcohol, a glycol, glycerol or acetone, or a mixture of these solvents. Preferably, it is an aqueous-glycolic extract. After maceration of the whole plant in a mixture of water and butylene glycol, the extract is filtered. An orangey-brown liquid with a characteristic odor, having the following analytical characteristics, is obtained:

pH: 3.5-5.5

density: 1.025-1.050

refractive index: 1.385-1.415

solids content: 2.0-6.0%.

An extract of Alchemilla vulgaris which can be used according to the invention can be obtained, for example, from the company Sederma, said extract being sold under the trade name “Alchemille BG”.

According to the invention, the extract of Alchemilla vulgaris can be used with one or more other depigmenting active agents. More particularly, the extract of Alchemilla vulgaris according to the invention can be combined, in a cosmetic or dermatological composition, with an extract of parsley, and/or an extract of chamomile, as described in patent FR 2 787 710.

The composition according to the invention contains of the order of 0.01 to 10% by weight, and preferably 0.01 to 5% by weight, of an extract of Alchemilla vulgaris in the form of a solution having a concentration ranging from 0.5 to 10% with respect to solids.

The cosmetic composition of the present invention for topical application can constitute in particular a cosmetic or dermatological protection, treatment or care composition for the face, for the neck, for the hands or for the body, such as, for example, day creams, night creams or body milks.

The cosmetic composition according to the present invention can contain one or more other components known to those skilled in the art, such as formulation agents or additives known to be conventionally used in cosmetic compositions. By way of example, and in a non-limiting manner, such formulation agents and additives may be hydrophilic or lipophilic gelling agents, softeners, dyes, solubilizing agents, texturing agents, fragrances, fillers, film-forming active agents, preserving agents, surfactants, emulsifiers, oils, glycols, vitamins, sunscreens, etc. By virtue of their knowledge in terms of cosmetics, those skilled in the art will know which formulation agents to add to the cosmetic composition according to the invention, and in what amounts according to the desired properties.

Advantageously, the composition according to the invention can contain one or more anti-UVA or anti-UVB sunscreens of synthetic or natural origin. Advantageously, the composition according to the invention can also contain one or more anti-aging active agents, in particular moisturizing active agents, antiwrinkle active agents, and tensioning agents.

In addition, the cosmetic composition according to the present invention can be in any form known to those skilled in the art in the cosmetics field, without any other specific pharmaceutical restriction other than that for application to the skin. Thus, the cosmetic composition according to the invention can be in the form of an aqueous or alcoholic solution or suspension, of an oily suspension, of a solution or a dispersion of lotion or serum type, of an emulsion with a liquid or semi-liquid consistency of milk type, obtained by dispersion of a fatty phase in an aqueous phase (oil-in-water emulsion: O/W) or conversely (water-in-oil: W/O), of an emulsion of the O/W or W/O cream type, of a gel, of a lotion or of a mask. The cosmetic formulations according to the invention can also be envisioned in the form of a mousse or else in the form of aerosol compositions also comprising a pressurized propellant.

The present invention also relates to a cosmetic treatment method for depigmenting the skin and/or for preventing the appearance of a pigmentation, comprising the application to the skin of a cosmetic composition containing an extract of Alchemilla vulgaris.

The present invention also relates to the use of an extract of Alchemilla vulgaris, for the preparation of a dermatological composition for use in preventing or treating skin hyperpigmentation.

In individuals who have a colored skin, such as Asians, for example, the use of an extract of Alchemilla vulgaris according to the present invention makes it possible to obtain and to conserve a lighter complexion, homogeneously over the entire area treated. In individuals who have a skin hyperpigmentation in the form of spots, of pathological or natural origin, the use of an extract of Alchemilla vulgaris makes it possible to restore the homogeneity of the complexion, by means of a lightening of the hyperpigmented areas.

The examples hereinafter relate, firstly, to the evaluation of the inhibition, with an extract of Alchemilla vulgaris, of melanosome transfer from the melanocyte to the keratinocyte, and, secondly, to the compositions which are the subjects of the present invention.

### I: EVALUATION OF THE INHIBITION, BY AN EXTRACT OF ALCHEMILLA VULGARIS, ON MELANOSOME TRANSFER

### A. Principle

The KGFR constitutes the keratinocyte receptor for KGF, the growth factor involved in epidermal proliferation and differentiation. This receptor is internalized when KGF binds. It has been shown that the KGFR has a role to play in melanosome transfer from melanocytes to keratinocytes. In fact, under the effect of KGF, said melanosomes enter the cell more readily. The melanosomes colocalize with the KGFR in internalization vesicles. This technique makes it possible to demonstrate the effect of certain molecules on the specific pathway of transfer via activation with KGF. It involves the use of fluospheres which mimic melanosomes on a cell-based study model of human keratinocytes in a monolayer (J. Invest. Dermatol 125: 1190-1199, 2005).

### B. Materials and Methods

### 1. Seeding

The human keratinocytes are cultured, on a glass coverslip in wells of a 6-well plate, in a proportion of 40×10³ cells/3 ml per well, at 37° C., in a DMEM medium (Dulbecco's modified essential medium) containng 2 mM of L-glutamine and 10% of fetal calf serum (FCS), in a moisture-saturated atmosphere with 5% of CO₂. The cultures are incubated for 72 hours.

### 2. Treatments

The cells are rinsed and incubated for 48 hours as follows:
Medium alone (control)
Extract of Alchemilla vulgaris 0.05%*
Extract of Alchemilla vulgaris 0.1%*
Extract of Alchemilla vulgaris 0.2%*
In the medium, final volume 3 ml, concentration found to be noncytotoxic for the cells in the Formazan blue reduction test.

### 3. Treatments and Incorporation of the Fluospheres by the Cells

The medium for the beads and the treatments for this step is different from that used above: DMEM, 10% inactivated FCS, 0.5% BSA (bovine serum albumin).

a) Preparation of the Fluorescent Beads

The beads are prepared in the above culture medium. The use of bovine serum albumin makes it possible to facilitate the homogeneous distribution of the beads in the solution. The inactivated fetal calf serum provides fewer nutrients and growth factors which can interact with the KGF provided by the experiment.

A concentration of 4×109 particles/ml is used for the experiment. The flask containing the beads is vortexed upon being removed from, the refrigerator, and then placed in the ultrasound bath for a period of 20 minutes. The beads are again vigorously vortexed and the amount required to prepare the working solution is removed (under a hood) and suspended in the medium. The working solution is subsequently vortexed and then immersed in the ultrasound bath for 10 minutes.

These steps are necessary in order to obtain a solution of beads which is as homogeneous as possible.

b) Preparation of the Treatments

The working solution of beads is distributed into various 15 ml tubes corresponding to the various concentrations of the treatment to be applied. The KGF (10 mg/ml) is added to each of the tubes in order to obtain a final concentration of 20 ng/ml. Each solution is mixed by suctioning in and out of a pipette.

c) Phagocytosis

A volume of 3 ml of each solution containing the KGF, the fluorescent beads and the extract of Alchemilla vulgaris at various concentrations (0.05; 0.1; 0.2%) is deposited in each well. A positive control well containing KGF alone is provided.

The plates are agitated by manual rotation in order to homogeneously distribute the solution. The cells are incubated for 2 h in the incubator at 37° C.-5%CO₂, the amount of time necessary for the KGFR to be internalized under the effect of the KGF.

d) Arrest of Phagocytosis

When the incubation period has elapsed, the incorporation of the beads by the cells must be stopped by removing the beads which have remained in the extracellular state. For this:
the entire content of each well is removed with the suction system;
1 ml of inactivated FCS is deposited in each well, it is agitated and placed in the incubator at 37° C.-5%CO₂, for a minimum of 20 minutes;
the FCS is removed and 4 vigorous rinses of approximately 1 minute are carried out with 2 to 3 ml of cold complete DMEM, the plates being agitated at each rinse;
2 rinses with phosphate buffered saline (PBS) are then carried out, by agitating the plates thoroughly and/or by thoroughly suctioning the PBS during the final rinse.

### 3. Visualization of the Transfer

The visualization is carried out on the same day. After the removal of the extracellular beads, the cells are fixed (methanol, −20° C.) on the cover slips in order to visualize the fluospheres, and also the keratinocytes by labeling the cytokeratin by means of the immunocytochemical technique.

For the labeling, the primary antibody used is rabbit anti-human cytokeratin IgG; the secondary antibody is FITC-coupled goat anti-rabbit IgG. A fluorescence microscope coupled to image analysis software allows us to visualize the labeling and to take photos.

### II. EXAMPLES

LIGHTENING CREAM

| | % |
|---|---|
| XANTHAN GUM | 0.10 |
| GLYCEROL | 5.00 |
| CETEARYL GLUCOSIDE | 3.00 |
| AE GLYCEROL MONOSTEARATE | 2.00 |
| C₈C₁₀ TRIGLYCERIDE | 10.00 |
| SILICONE OIL | 3.00 |
| EXTRACT OF PARSLEY | 1.00 |
| EXTRACT OF CHAMOMILE | 1.00 |
| EXTRACT OF ALCHEMILLA VULGARIS | 2.00 |
| PRESERVING AGENTS | 1.00 |
| FRAGRANCE | 0.30 |
| DEMINERALIZED WATER | Q.S. 100 |

LIGHTENING GEL

| | % |
|---|---|
| GLYCEROL | 3.00 |
| XANTHAN GUM | 0.20 |
| ETHANOL | 5.00 |
| EXTRACT OF PARSLEY | 1.00 |
| EXTRACT OF CHAMOMILE | 1.00 |
| EXTRACT OF ALCHEMILLA VULGARIS | 2.00 |
| PRESERVING AGENTS | 0.50 |
| SOLUBILIZING AGENT | 0.50 |
| FRAGRANCE | 0.20 |
| DEMINERALIZED WATER | Q.S. 100 |

LIGHTENING LOTION

| | % |
|---|---|
| GLYCOL | 2.00 |
| SODIUM CHLORIDE | 1.00 |
| ETHANOL | 5.00 |
| EXTRACT OF PARSLEY | 1.00 |
| EXTRACT OF CHAMOMILE | 1.00 |
| EXTRACT OF ALCHEMILLA VULGARIS | 2.00 |
| PRESERVING AGENTS | 0.50 |
| SOLUBILIZING AGENT | 0.30 |
| FRAGRANCE | 0.10 |
| DEMINERALIZED WATER | Q.S. 100 |

1. A composition comprising an extract of Alchemilla vulgaris in a depigmenting cosmetic composition or in a cosmetic composition as a depigmenting agent, or in a dermatological composition for use in preventing or treating skin hyperpigmentation.

2. (canceled)

3. The composition as claimed in claim 1, wherein the extract of Alchemilla vulgaris is derived from a whole plant.

4. The composition as claimed in claim 1, wherein the extract of Alchemilla vulgaris is an aqueous-glycolic extract.

5. The composition as claimed in claim 1, wherein the composition contains of an order of 0.01 to 10% by weight, of an extract of Alchemilla vulgaris in a form of a solution having a concentration ranging from 0.5 to 10% with respect to solids.

6. The composition as claimed in claim 1, wherein the composition also contains an extract of parsley.

7. The composition as claimed in claim 1, wherein the composition also contains an extract of chamomile.

8. A cosmetic treatment method for depigmenting the skin, comprising application to skin of a cosmetic composition according to claim 1, containing said extract of Alchemilla vulgaris.

9. The composition of claim 1, comprising said dermatological composition for use in preventing or treating skin hyperpigmentation.

10. The composition of claim 1, comprising said depigmenting cosmetic composition.

11. The composition of claim 1, comprising said cosmetic composition as a depigmenting agent.

12. The composition of claim 5 containing about 0.01-5% by weight said extract.
